(19) United States
(12) Patent Application Publication
(10) Pub. No.: US 2022/0047222 A1
(43) Pub. Date: Feb. 17, 2022

(54) METHOD FOR DETERMINING INJURY RISK OF USER TAKING EXERCISE

(71) Applicant: bOMDIC, Inc., New Taipei (TW)

(72) Inventors: MING-CHIA YEH, New Taipei (TW); SZU-HONG CHEN, New Taipei (TW)

(21) Appl. No.: 16/990,985

(22) Filed: Aug. 11, 2020

(51) Int. Cl. A61B 5/00; A61B 5/0205; A63B 24/00; G16H 20/30; G16H 50/30

(52) U.S. Cl. CPC A61B 5/7275; A61B 5/0205; A61B 5/024; G16H 20/30; G16H 50/30; A63B 24/0062

(57) ABSTRACT

The present invention discloses a method for determining an injury risk of a user who has performed an exercise training for a first duration. Divide the first duration into a plurality of time segments. Determine the training load in each of the plurality of time segments. A first portion of the training load is above a threshold of an exercise intensity adjusted according to a fitness condition of the user. Perform an algorithm to determine an indication mode representing a training condition of the exercise training of the user in the first duration based on the training load. Determine a criterion of the injury risk based on the first portion of the training load and the algorithm. Determine the injury risk of the user based on a comparison between the indication mode and the criterion of the injury risk.

### BACKGROUND OF THE INVENTION

### 1. Field of the Invention

The present invention relates to a method for monitoring an exercise of a user, and more particularly to a method for determining an injury risk of a user taking exercise.

### 2. Description of Related Art

Overtraining may increase injury risk in an exercise session; therefore, monitoring training load is very important in preventing the user from getting injured. Recently, ACWR (Acute Chronic Workload Ratio) is a parameter used for estimating the injury risk of the exercise. This parameter is a ratio of the accumulated training load in the short-term to the accumulated training load in the long-term.

However, this parameter doesn't take the fitness condition (e.g., fitness performance level; the parameter of the fitness performance level is preferably VO₂ₘₐₓ) of the user into account. Take this case for example. The paper indicates that there is more injury risk as the positive value, equal to 1.5 subtracted from ACWR, is more. If the user A of more fitness performance level and the user B of less fitness performance level having the same age as the user A (i.e., the user A and the user B have the same age-based heart-rate zones. The age-based heart-rate zones is determined as follows: first, compute the maximum heart rate according to the formula, such as 220 minus the user's age (unit: beats per minute (BPM)); second, each of the personalized heart-rate zones is determined according to the ratio range of the maximum heart rate, and the ratio range is based on the common knowledge in the exercising field.) have performed the same exercise training for 28 days (i.e., TRIMP (training impulse) of the user A is the same as TRIMP of the user B everyday), ACWR of the user A is also the same as ACWR of the user B. At this time, if ACWR of the user A and ACWR of the user B are both 1.6, the user B of less fitness performance level may feel injury risk but the user A of more fitness performance level may feel no injury risk; further, the user A of more fitness performance level may still want to increase exercise intensity and continuously takes exercise to further improve his fitness performance level; however, a hint from ACWR may make the user A of more fitness performance level lose motivation for training psychologically.

Accordingly, the present invention proposes a method for determining an injury risk of a user taking exercise to overcome the above-mentioned disadvantages.

### SUMMARY OF THE INVENTION

In the present invention, determine the injury risk of the user who has performed the exercise training for a first duration based on a comparison between the indication mode representing the training condition and the criterion of the injury risk. The criterion of the injury risk is determined based on the high-exercise-intensity training load (i.e., a first portion of the training load above the threshold of the exercise intensity) adjusted according to the fitness condition of the user.

In the present invention, the threshold of the exercise intensity adjusted according to the fitness condition of the user is a technical feature which takes the fitness condition (e.g., fitness performance level) of the user into account. In other words, the different criterion of the injury risk is determined for the users with different fitness conditions based on the high-exercise-intensity training load (i.e., a first portion of the training load above the threshold of the exercise intensity) adjusted according to the fitness condition of the user by this technical feature. Once the fitness condition of the user is determined, the criterion of the injury risk for the user can be precisely determined and the injury risk of the user who has performed the exercise training for the first duration can be precisely determined by this technical feature.

In a preferred embodiment of the present invention, the training load is determined based on a plurality of exercise intensity zones adjusted based on the fitness condition (e.g., fitness performance level) of the user. Besides the criterion of the injury risk determined in the preceding paragraph takes the fitness condition (e.g., fitness performance level) of the user into account, the indication mode determined based on at least one parameter associated with the training load also takes the fitness condition (e.g., fitness performance level) of the user into account because the training load is determined based on a plurality of exercise intensity zones adjusted based on the fitness condition (e.g., fitness performance level) of the user. Once the fitness condition of the user is determined, the injury risk of the user who has performed the exercise training for the first duration can be precisely determined based on the comparison between the indication mode representing the training condition and the criterion of the injury risk.

By the algorithm implemented in the computer of the present invention, the computer of the present invention performs operations described in claims or the following descriptions to precisely the injury risk of the user who has performed the exercise training for the first duration.

In one embodiment, the present invention discloses a method for determining an injury risk of a user who has performed an exercise training for a first duration. The method comprises: dividing, by a processing unit, the first duration into a plurality of time segments; determining, by the processing unit, the training load in each of the plurality of time segments, wherein a first portion of the training load is above a threshold of an exercise intensity adjusted according to a fitness condition of the user; performing, by the processing unit, an algorithm to determine an indication mode representing a training condition of the exercise training of the user in the first duration based on at least one first parameter associated with the training load; determining, by the processing unit, a criterion of the injury risk based on at least one second parameter associated with the first portion of the training load and the algorithm determining the indication mode; and determining, by the processing unit, the injury risk of the user who has performed the exercise training for the first duration based on a comparison between the indication mode representing the training condition and the criterion of the injury risk.

In one embodiment, the present invention discloses a method for determining an injury risk of a user who has performed an exercise training for a first duration. The method comprises: dividing, by a processing unit, a first duration into a plurality of time segments; determining, by the processing unit, the training load in each of the plurality of time segments, wherein the training load is determined based on a plurality of exercise intensity zones, wherein each of the plurality of exercise intensity zones has a first portion of the training load, wherein the training load is a sum of the first portions of the plurality of exercise intensity zones, wherein a second portion of the training load is above a threshold of an exercise intensity, wherein the plurality of exercise intensity zones and the threshold of the exercise intensity are adjusted according to the fitness condition of the user; performing, by the processing unit, an algorithm to determine an indication mode representing a training condition of the exercise training of the user in the first duration based on at least one first parameter associated with the training load; determining, by the processing unit, a criterion of the injury risk based on at least one second parameter associated with the second portion of the training load; and determining, by the processing unit, the injury risk of the user who has performed the exercise training for the first duration based on a comparison between the indication mode representing the training condition and the criterion of the injury risk.

The detailed technology and above preferred embodiments implemented for the present invention are described in the following paragraphs accompanying the appended drawings for people skilled in the art to well appreciate the features of the claimed invention.

### BRIEF DESCRIPTION OF THE DRAWINGS

The foregoing aspects and many of the accompanying advantages of this invention will become more readily appreciated as the same becomes better understood by reference to the following detailed description when taken in conjunction with the accompanying drawings, wherein:

FIG. 1 illustrates a schematic block diagram of an exemplary apparatus in the present invention;

FIG. 2 illustrates a method for determining an injury risk of a user who has performed an exercise training for a first duration;

FIG. 3A illustrates the training load (TL) per day in one embodiment the present invention;

FIG. 3B illustrates the training load (TL) of higher exercise intensity per day in one embodiment the of present invention;

FIG. 3C illustrates the training load (TL) of lower exercise intensity per day in one embodiment the present invention;

FIG. 4A illustrates the relationship between the fitness performance level of the user and the threshold of the exercise intensity in a table for adjustment;

FIG. 4B illustrates that the fitness performance level of the user may be ranked such that each fitness performance level has a corresponding threshold of the exercise intensity;

FIG. 5A illustrates the two-dimensional exercise intensity zones;

FIG. 5B illustrates the one-dimensional exercise intensity zones derived from the two-dimensional exercise intensity zones in FIG. 5A; and

FIG. 6 illustrates that the criterion of the injury risk varies with exercise time.

### DETAILED DESCRIPTION OF THE ILLUSTRATED EMBODIMENTS

The detailed explanation of the present invention is described as following. The described preferred embodiments are presented for purposes of illustrations and description and they are not intended to limit the scope of the present invention.

### Definition of the Terms

Fitness Condition

The fitness condition may be defined by the fitness performance level. The fitness performance level of one user may be different from that of the other user; if two users want to achieve the same training effect, one user of more fitness performance level needs acuter exercise guiding and higher exercise intensity than the other user of less fitness performance level. The fitness performance level may include health-related fitness and sport/skill-related fitness which can be also improved by engaging in physical activities or exercise training. For example, the parameter of the fitness performance level may be VO₂ₘₐₓ or METₘₐₓ (maximum oxygen uptake capacity relative to resting oxygen consumption: equal to VO₂ₘₐₓ/3.5), and VO₂ₘₐₓ is preferred. Generally, a unit of the VO₂ₘₐₓ can be represented in an absolute way, such as oxygen uptake (ml/min), or in a relative way, such as oxygen uptake based on weight (ml/kg/min).

Exercise Intensity

The exercise intensity may refer to how much energy is expended when exercising. The exercise intensity may define how hard the body has to work to overcome a task/exercise. Exercise intensity may be measured in the form of the internal workload. The parameter of the exercise intensity associated with the internal workload may be associated with a heart rate, an oxygen consumption, a pulse, a respiration rate and RPE (rating perceived exertion). The exercise intensity may be measured in the form of the external workload. The parameter of the exercise intensity associated with the external workload may be associated with a speed, a power, a force, a motion intensity, a motion cadence or other kinetic data created by the external workload resulting in energy expenditure. The heart rate may be often used as a parameter of the exercise intensity.

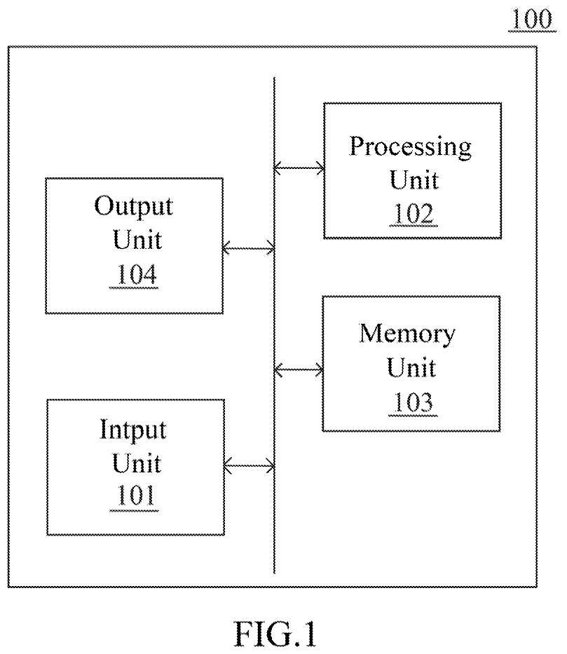

The method in the present invention can be applied in all kinds of apparatuses, such as an exercise measurement system, a wrist top device, a mobile device, a server or a combination of at least one of the exercise measurement system, the wrist top device, the mobile device and the server. FIG. 1 illustrates a schematic block diagram of an exemplary apparatus 100 in the present invention. The apparatus 100 may comprise an input unit 101, a processing unit 102, a memory unit 103 and an output unit 104. The input unit 101 may comprise a first sensor which may measure the exercise intensity associated with the physiological data, the cardiovascular data or the internal workload from the user's body. The exercise intensity may be measured by applying a skin contact from chest, wrist or any other human part. Preferably, the exercise intensity is a heart rate and the sensor is a heart rate senor. The input unit 101 may comprise a second sensor (e.g., motion sensor) which may measure the exercise intensity associated with the external workload. The second sensor may comprise at least one of an accelerometer, a magnetometer and a gyroscope. The input unit 101 may further comprise a position sensor (e.g., GPS: Global Positioning System). In the present invention, the exercise-associated parameter (e.g., training load) is calculated based on the exercise intensity measured by a sensor. The processing unit 102 may be any suitable processing device for executing software instructions, such as a central processing unit (CPU). The memory unit 103 may include random access memory (RAM) and read only memory (ROM), but it is not limited to this case. The memory unit 103 may include any suitable non-transitory computer readable medium, such as ROM, CD-ROM, DVD-ROM and so on. Also, the non-transitory computer readable medium is a tangible medium. The non-transitory computer readable medium includes a computer program code. The memory unit 103 and the computer program code are configured with the processing unit 102 to cause the apparatus 100 to perform desired operations (e.g., operations listed in claims). The output unit 104 may be a display for displaying exercise guiding, exercise scheme or exercise index. The displaying mode may be in the form of words, a voice or an image.

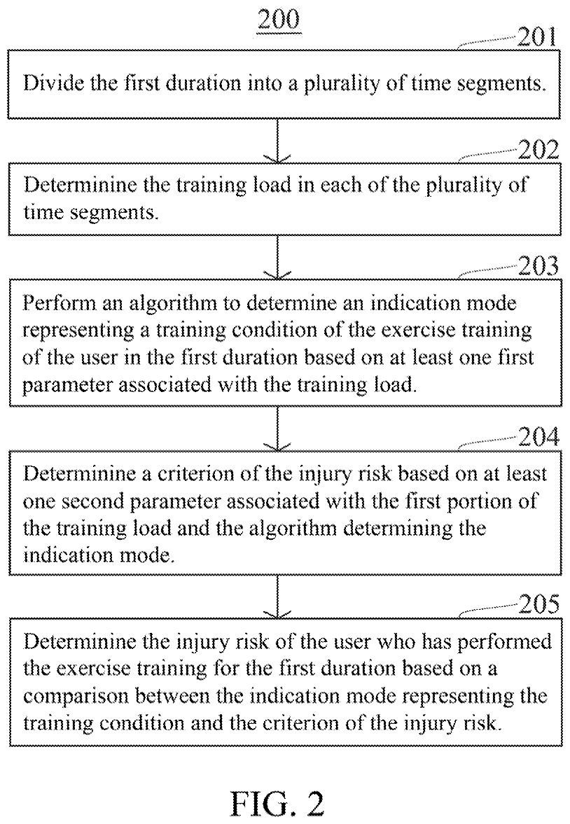

FIG. 2 illustrates a method 200 for determining an injury risk of a user who has performed an exercise training for a first duration. The process in FIG. 2 starts in step 201: dividing, by the first duration into a plurality of time segments (by the processing unit 102). Each time segment may have the same period or a different period. Preferably, each time segment may have the same period. For convenience of description, the first duration is 28 days; however, the present invention is not limited to this case.

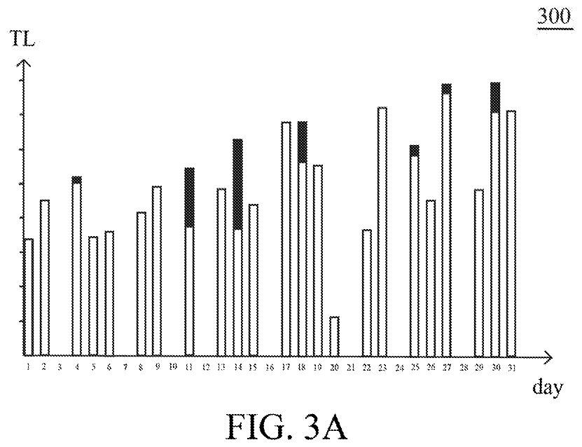

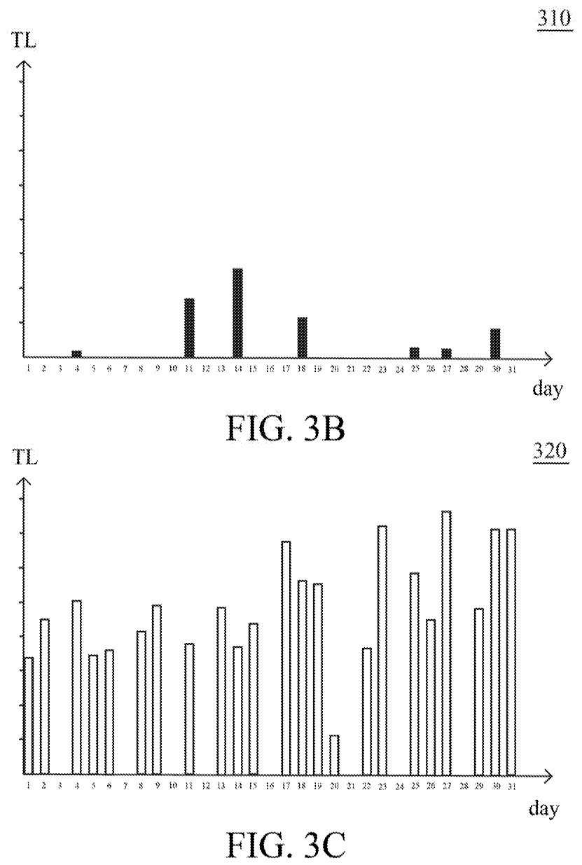

In Step 202: determining the training load in each of the plurality of time segments (by the processing unit 102). For convenience of description, the period of each time segment is 1 day in the present invention; however, the present invention is not limited to this case. In the present invention, the training load is calculated everyday. FIG. 3A illustrates the training load (TL) per day 300 in one embodiment the present invention. In the following description, the criterion of the injury risk is determined based on the training load of higher exercise intensity; therefore, the training load of higher exercise intensity (see the portion in black) is defined as a portion of the training load is above the threshold of the exercise intensity. FIG. 3B illustrates the training load (TL) of higher exercise intensity per day 310 in one embodiment the of present invention. The training load of higher exercise intensity per day is above the threshold of the exercise intensity in FIG. 3B. FIG. 3C illustrates the training load (TL) of lower exercise intensity per day 320 in one embodiment the present invention. The training load of lower exercise intensity per day is below the threshold of the exercise intensity in FIG. 3C. The training load per day in FIG. 3A is a sum of the training load of higher exercise intensity per day in FIG. 3B and the training load of lower exercise intensity per day in FIG. 3C.

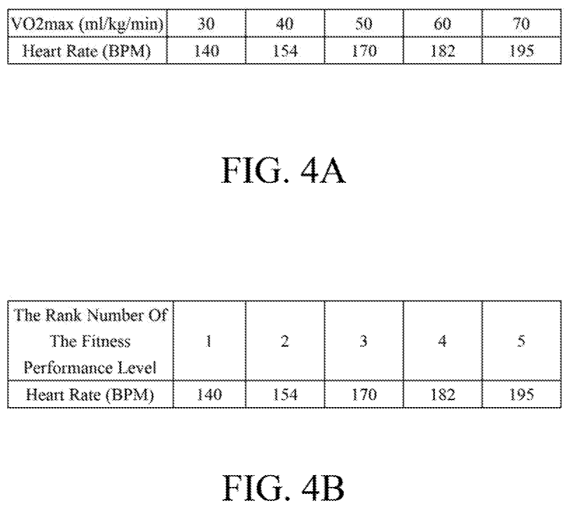

The threshold of the exercise intensity is adjusted according to the fitness condition (e.g., fitness performance level; the parameter of the fitness performance level is preferably VO₂ₘₐₓ) of the user. In other words, the threshold of the exercise intensity is adjusted based on the different fitness performance levels. FIG. 4A illustrates the relationship between the fitness performance level of the user and the threshold of the exercise intensity in a table for adjustment. In FIG. 4A, the parameter of the fitness performance level is VO₂ₘₐₓ and the parameter of the threshold of the exercise intensity is a heart rate. The relationship may be acquired by a statistics analysis of big data. If VO₂ₘₐₓ of the user doesn't exist in this table, the threshold of the exercise intensity for VO₂ₘₐₓ of the user can be determined by an interpolation method or an extrapolation method. FIG. 4B illustrates that the fitness performance level of the user may be ranked such that each fitness performance level has a corresponding threshold of the exercise intensity. In FIG. 4B, the fitness performance level is ranked such that the higher fitness performance level has a larger rank number and the parameter of the threshold of the exercise intensity is a heart rate. The fitness condition of the user may be determined by taking U.S. application Ser. No. 16/843,853 as a reference. For example, the parameter representing the fitness condition may be a gradient of an accumulating physiological index, such the descending rate of the fitness index or stamina index. The parameter representing the fitness condition may be a gradient of a physiological index, such as the ascending rate of the physiological data (e.g., heart rate). A combination of the multiple parameters may represent the fitness condition. The combination of the multiple parameters may be in the form of a synthetic index (e.g., weighed average, e*P+f*Q). As the fitness condition of the user is changed by monitoring a physiological data, cardiovascular data or the internal workload measured by the exercise measurement system (or a sensor), the threshold of the exercise intensity is also changed. Generally, the threshold of the exercise intensity is adjusted to be larger as the fitness condition of the user is improved.

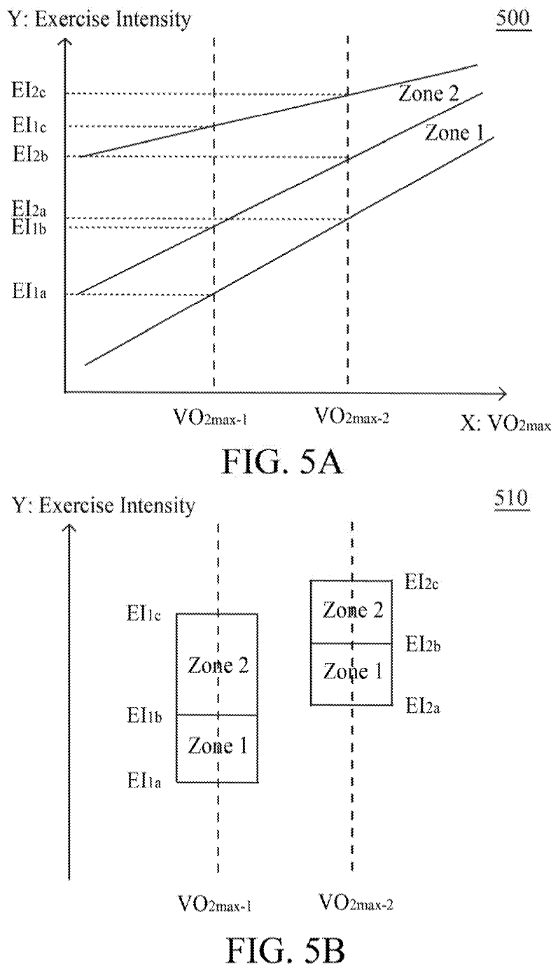

The training load may be determined based on a plurality of exercise intensity zones. Each of the exercise intensity zones has a second portion of the training load (e.g., the product of the exercise intensity and the exercise time), wherein the training load is a sum of the second portions of the exercise intensity zones. At least one of the exercise intensity zones is adjusted according to the fitness condition of the user or adjusted based on the different fitness performance levels. All of the exercise intensity zones are adjusted according to the fitness condition of the user or adjusted based on the different fitness performance levels. In one embodiment, the training load is determined based on a plurality of exercise intensity zones in U.S. application Ser. No. 16/733,180 which can be incorporated by reference therein. Obviously, U.S. application Ser. No. 16/733,180 discloses a plurality of exercise intensity zones adjusted according to the fitness condition (e.g., fitness performance level) of the user or adjusted based on the different fitness performance levels (see two-dimensional exercise intensity zones 500 in FIG. 5A and one-dimensional exercise intensity zones 510 in FIG. 5B); the details can be seen in the corresponding description of U.S. application Ser. No. 16/733,180. The threshold of the exercise intensity is a lower boundary of one exercise intensity zone having the highest exercise intensity range of the plurality of exercise intensity zones (If the parameter of the exercise intensity is a heart rare, the upper boundary of one exercise intensity zone having the highest exercise intensity range of the plurality of exercise intensity zones may be maximum heart rate).

In one embodiment, the training load may be represented in the form of an TRIMP (training impulse); however, the present invention is not limited to this case.

In step 203: performing an algorithm to determine an indication mode representing a training condition of the exercise training of the user in the first duration based on at least one first parameter Wᵢ associated with the training load (by the processing unit 102). To understand the training condition (e.g., training time or training distribution) of the exercise training of the user in the first duration, the indication mode may be determined by performing an algorithm. In one embodiment, one of at least one parameter Wᵢ may be further associated with the accumulated training load. The parameter Wᵢ may be presented in a relative way or in an absolute way by using the accumulated training load as the input of the parameter Wᵢ. The algorithm may adopt the parameter Wᵢ presented in an absolute way, such as the accumulated training load during the past several days or 28 days. The algorithm may adopt the parameter Wᵢ presented in a relative way, such as the ratio of the short-term accumulated training load to the long-term accumulated training load (e.g., ACWR: Acute Chronic Workload Ratio); the long-term may be a first duration (e.g., 28 days) and the short-term may be a second duration (e.g., 7 days).

In a further embodiment, the indication mode may be determined further based on at least one parameter Vᵢ associated with a recover condition (e.g., recovery time or recovery distribution). The recover condition may comprise a succession of time segments each of which doesn't have the training load therein (i.e., the training load is 0). For example, the algorithm may adopt a combination of the parameter Wᵢ and the parameter Vᵢ, such as a weighed synthetic index (e.g., a*Wᵢ+b*Vᵢ, each of the coefficients a, b may be fixed or variable according to the observation of the physiological phenomenon) of the parameter Wᵢ and the parameter Vᵢ; the parameter Vᵢ associated with the recover condition may be a parameter V₁ representing the number of a succession of days each of which has no training load therein before the current training load or a parameter V₂ representing the number of the days each of which has no training load therein in a duration before the current training load. For example, see FIG. 3A, if the current training load happens on day No. 8, the number of a succession of days each of which has no training load is 1 (i.e., day No. 7); if the current training load happens on day 13, the number of the days of the duration before the current training load is 10 (i.e., days No. 3-12), the number of the days each of which has no training load is 4 (i.e., days No. 3, 7, 10, 12).

In Step 204: determining a criterion of the injury risk based on at least one second parameter Xᵢ associated with the first portion of the training load and the algorithm determining the indication mode (by the processing unit 102). The criterion of the injury risk may be dynamically determined. The criterion of the injury risk may be determined relative to a predetermined criterion associated with the algorithm. The predetermined criterion may be fixed. For example, the paper indicates that there is more injury risk as the positive value, equal to 1.5 subtracted from ACWR, is more and then the predetermined criterion may be 1.5 when the algorithm only adopts ACWR (Acute Chronic Workload Ratio) to represent a training condition of the exercise training of the user in the first duration in step 203. In another example, the predetermined criterion may be variable. Furthermore, the predetermined criterion may be user-defined.

The parameter X₁ associated with the first portion of the training load may be presented in an absolute way, such as the current first portion of the training load. The parameter X₂ associated with the first portion of the training load may be presented in a relative way, such as the ratio of the current first portion of the training load to the current overall training load. The parameter X₃ associated with the first portion of the training load may be the number of the days each of which has the first portion of the training load therein in a duration before the current first portion of the training load. For example, see FIG. 3B; if the current first portion of the training load happens on day No. 18, the number of the days of the duration before the current first portion of the training load is 17 (i.e., days No. 1-17), the number of the days each of which has the first portion of the training load therein is 3 (i.e., days 4, 11, 14).

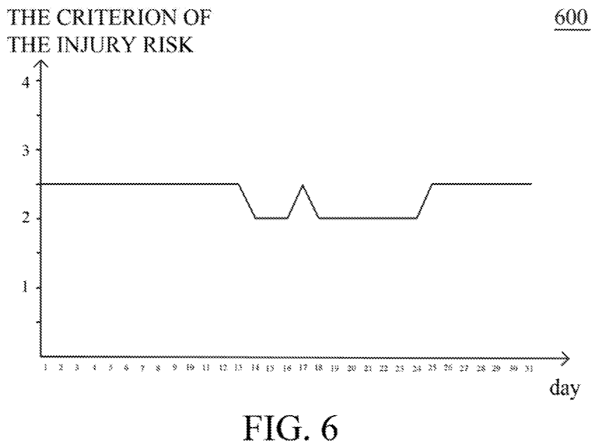

In one embodiment, when the algorithm only adopts ACWR (Acute Chronic Workload Ratio) to represent a training condition of the exercise training of the user in the first duration in step 203, the criterion of the injury risk may be determined by using: (1) the criterion of the injury risk=function f(X₁)=c1*X₁; or (2) the criterion of the injury risk=function f(X₂)=c2*X₂; or (3) the criterion of the injury risk (a combination of X₂ and X₃)=function f(X₂, X₃)=c3*X₂+c4*X₃. Each of the coefficients c1, c2, c3, c4 may be fixed or variable according to the observation of the physiological phenomenon. Take the case (I) for example: the criterion of the injury risk (a combination of X₂ and X₃)=function f(X₂, X₃)=c3*X₂+c4*X₃; each of the coefficients c3, c4 is positive. The more the parameter X₂ is, the less the criterion of the injury risk is adjusted to be, which will increase the injury risk. The more the parameter X₃, the less the criterion of the injury risk is adjusted to be, which will increase the injury risk. The more the parameter X₂ and the parameter X₃ are both at the same time, the less the criterion of the injury risk adjusted to be, which will increase the injury risk. FIG. 6 illustrates that the criterion of the injury risk 600 varies with exercise time in this example.

Further, the criterion of the injury risk may be determined further based on at least one parameter Yᵢ associated with a recover condition. The recover condition comprises a succession of time segments each of which doesn't have the first portion of the training load therein (i.e., the first portion of the training load is 0). The parameter Yᵢ associated with the recover condition may be a parameter Yᵢ representing the number of a succession of days each of which has no first portion of the training load therein before the current first portion of the training load or a parameter Y₂ representing the number of the days each of which has no first portion of the training load therein in a duration before the current first portion of the training load. For example, see FIG. 3B, if the current first portion of the training load happens on day No. 11, the number of a succession of days each of which has no first portion of the training load is 6 (i.e., days No. 5-10); if the current first portion of the training load happens on day No. 14, the number of the days of the duration before the current first portion of the training load 10 (i.e., days No. 4-13), the number of the days each of which has no first portion of the training load is 8 (i.e., days No. 5-10, 12-13).

In one embodiment, when the algorithm only adopts ACWR (Acute Chronic Workload Ratio) to represent a training condition of the exercise training of the user in the first duration in step 203, the criterion of the injury risk may be determined by using the criterion of the injury risk (a combination of X₂ and Y₁)=function f(X₂,Y₁)=c5*X₂=c6*Y₁. Each of the coefficients c5, c6 may be fixed or variable according to the observation of the physiological phenomenon. For example, each of the coefficients c5, c6 is positive. The more the parameter X₂ is, the less the criterion of the injury risk is adjusted to be, which will increase the injury risk. The more the parameter Y₁ is, the more the criterion of the injury risk is adjusted to be, which will decrease the injury risk. When the parameter X₂ and the parameter Yᵢ both increase at the same time, the criterion of the injury risk depends on the competence of the parameter X₂ and the parameter Yᵢ of the function f(X₂, Yᵢ).

Finally, in Step 205: determining the injury risk of the user who has performed the exercise training for the first duration based on a comparison between the indication mode representing the training condition and the criterion of the injury risk (by the processing unit 102). Take the case (I) for example: the criterion of the injury risk (a combination of X₂ and X₃)=function f(X₂, X₃)=c3*X₂+c4*X₃; each of the coefficients c3, c4 is positive. FIG. 6 illustrates that the criterion of the injury risk is between 2-2.5 for the user A of more fitness performance level. Even if ACWR of the user A is 1.6 more than 1.5 in the paper, it is still lower than the criterion of the injury risk between 2-2.5 because the user A has more fitness performance level. The user A of more fitness performance level may still increase exercise intensity and continuously takes exercise until ACWR reaches at least 2. Therefore, the user A of more fitness performance level still keeps motivation for training to further improve his fitness performance level.

In the above description, the indication mode and the criterion of the injury risk are numerically represented in the form of value or index. If the indication mode is less than the criterion of the injury risk, there is no injury risk or less injury risk; on the contrary, if the indication mode is more than the criterion of the injury risk, there is injury risk or more injury risk. The indication mode and the criterion of the injury risk may be presented in any suitable form. For example, the indication mode and the criterion of the injury risk are respectively presented in the form of the pattern 1 and in the form of the pattern 2; and then the comparison between the indication mode representing the training condition and the criterion of the injury risk may be based on the shift between the pattern 1 and the pattern 2.

The above disclosure is related to the detailed technical contents and inventive features thereof. People skilled in the art may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A method for determining an injury risk of a user who has performed an exercise training for a first duration, the method comprising:
dividing, by a processing unit, the first duration into a plurality of time segments;
determining, by the processing unit, the training load in each of the plurality of time segments, wherein a first portion of the training load is above a threshold of an exercise intensity adjusted according to a fitness condition of the user;
performing, by the processing unit, an algorithm to determine an indication mode representing a training condition of the exercise training of the user in the first duration based on at least one first parameter associated with the training load;
determining, by the processing unit, a criterion of the injury risk based on at least one second parameter associated with the first portion of the training load and the algorithm determining the indication mode; and
determining, by the processing unit, the injury risk of the user who has performed the exercise training for the first duration based on a comparison between the indication mode representing the training condition and the criterion of the injury risk.

2. The method according to claim 1, wherein the training load is determined based on a plurality of exercise intensity zones.

3. The method according to claim 2, wherein each of the plurality of exercise intensity zones has a second portion of the training load, wherein the training load is a sum of the second portions of the plurality of exercise intensity zones.

4. The method according to claim 1, wherein the training load is represented in the form of an TRIMP (training impulse).

5. The method according to claim 2, wherein the plurality of exercise intensity zones adjusted according to the fitness condition of the user.

6. The method according to claim 5, wherein the threshold of the exercise intensity is a lower boundary of one exercise intensity zone having the highest exercise intensity range of the plurality of exercise intensity zones.

7. The method according to claim 1, wherein the threshold of the exercise intensity is adjusted to be larger as the fitness condition of the user is improved.

8. The method according to claim 1, wherein the criterion of the injury risk is determined relative to a predetermined criterion associated with the algorithm.

9. The method according to claim 1, wherein a third parameter of the exercise intensity is associated with a heart rate, an oxygen consumption, a pulse, a respiration rate or RPE (rating perceived exertion).

10. The method according to claim 1, wherein a third parameter of the exercise intensity is associated with a speed, a power, a force, a motion intensity or a motion cadence.

11. The method according to claim 1, wherein the training load is calculated based on the exercise intensity measured by a sensor.

12. The method according to claim 11, wherein the exercise intensity is a heart rate and the sensor is a heart rate senor.

13. The method according to claim 11, wherein the exercise intensity is associated with an external workload and the sensor is motion senor.

14. The method according to claim 1, wherein one of the at least one first parameter is further associated with the accumulated training load.

15. The method according to claim 1, wherein the indication mode representing the training condition of the exercise training of the user in the first duration is determined further based on at least one third parameter associated with a recover condition.

16. The method according to claim 15, wherein the recover condition comprises a succession of time segments each of which doesn't have the training load therein.

17. The method according to claim 1, wherein the criterion of the injury risk is determined further based on at least one third parameter associated with a recover condition.

18. The method according to claim 17, wherein the recover condition comprises a succession of time segments each of which doesn't have the first portion of the training load therein.

19. An apparatus for determining an injury risk of a user who has performed an exercise training for a first duration, the apparatus comprising:
a processing unit; and
a memory unit including a computer program code, wherein the memory unit and the computer program code are configured, with the processing unit, to cause the apparatus to perform a process comprising steps of:
dividing, by the processing unit, the first duration into a plurality of time segments;
determining, by the processing unit, the training load in each of the plurality of time segments, wherein a first portion of the training load is above a threshold of an exercise intensity adjusted according to a fitness condition of the user;
performing, by the processing unit, an algorithm to determine an indication mode representing a training condition of the exercise training of the user in the first duration based on at least one first parameter associated with the training load;
determining, by the processing unit, a criterion of the injury risk based on at least one second parameter associated with the first portion of the training load and the algorithm determining the indication mode; and
determining, by the processing unit, the injury risk of the user who has performed the exercise training for the first duration based on a comparison between the indication mode representing the training condition and the criterion of the injury risk.

20. A method for determining an injury risk of a user who has performed an exercise training for a first duration, the method comprising:
dividing, by a processing unit, a first duration into a plurality of time segments;
determining, by the processing unit, the training load in each of the plurality of time segments, wherein the training load is determined based on a plurality of exercise intensity zones, wherein each of the plurality of exercise intensity zones has a first portion of the training load, wherein the training load is a sum of the first portions of the plurality of exercise intensity zones, wherein a second portion of the training load is above a threshold of an exercise intensity, wherein the plurality of exercise intensity zones and the threshold of the exercise intensity are adjusted according to the fitness condition of the user;
performing, by the processing unit, an algorithm to determine an indication mode representing a training condition of the exercise training of the user in the first duration based on at least one first parameter associated with the training load;
determining, by the processing unit, a criterion of the injury risk based on at least one second parameter associated with the second portion of the training load; and
determining, by the processing unit, the injury risk of the user who has performed the exercise training for the first duration based on a comparison between the indication mode representing the training condition and the criterion of the injury risk.
